(19) United States
(12) Patent Application Publication
(10) Pub. No.: US 2015/0299594 A1
(43) Pub. Date: Oct. 22, 2015

(54) PROCESS FOR THE PREPARATION OF HYDROCARBONS

(71) Applicant: HALDOR TOPSOE A/S, Kgs. Lyngby (DK)

(72) Inventors: Berit Hinnemann, Stenlose (DK); Arne Knudsen, Copenhagen (DK)

(21) Appl. No.: 14/437,706

(22) PCT Filed: Nov. 22, 2012
(86) PCT No.: PCT/EP2012/073346

§ 371 Date: Apr. 22, 2015

(30) Foreign Application Priority Data
Oct. 23, 2012 (DK) PA 2012 70645

(51) Int. Cl. C10L 1/06; C07C 29/151; C01B 3/36; C07C 29/153; C07C 41/09; C10G 3/00; C07C 29/154

(52) U.S. Cl. CPC C10L 1/06; C10G 3/49; C07C 29/1518; C07C 29/154; C07C 29/153; C07C 41/09; C01B 3/36; C10L 2270/023; C10L 2290/08; C10L 2290/10; C10L 2290/42; C01B 2203/0205; C01B 2203/061; C01B 2203/1276

(57) ABSTRACT

Process for the preparation of higher hydrocarbons boiling in the gasoline range from methane containing feed gas comprising the steps of a) mixing the feed gas with a hydrogenated tail gas and autothermal reforming the mixed feed gas to a methanol synthesis gas containing hydrogen, carbon monoxide and carbon dioxide; b) converting the methanol synthesis gas to a methanol and dimethyl ether containing effluent in presence of one or more catalysts active in the conversion of hydrogen and carbon oxides to methanol and dehydration of methanol to dimethyl ether; c) converting the methanol and dimethyl ether containing effluent as prepared in step (b) to a raw product containing hydrocarbons boiling in the gasoline range, water, unconverted methanol synthesis gas and carbon dioxide formed during the conversion of the methanol synthesis gas; d) cooling and separating the raw product into a water fraction, a higher hydrocarbon fraction boiling in the gasoline range and into a tail gas with the unconverted methanol synthesis gas and the carbon dioxide; e) hydrogenating a part of the tail gas as obtained in step d) to provide the hydrogenated tail gas; and f) recycling the hydrogenated tail gas to step (a).

The invention relates to a process for the preparation of hydrocarbons from gaseous fuels. In particular, the invention relates to the preparation of hydrocarbons useful as gasoline compounds from synthesis gas obtained from autothermal reforming of natural gas and/or coke oven gas.

Synthesis gas can be obtained in a variety of manners, for instance by reforming natural gas or other methane rich gases like coke oven gas or a mixture of coke oven gas and blast furnace gas.

As an example, a process for the preparation of chemical raw materials by steam reforming of a mixture of coke oven gas and blast furnace gas is mentioned in EP 0 200 880. The amount of coke oven gas and blast furnace gas is in this process adjusted and subjected to methanation in order to obtain a stoichiometric synthesis gas for the preparation of methanol (MeOH).

The synthetic gasoline process is known to take place in two steps: the conversion of synthesis gas to oxygenates and the conversion of oxygenates to gasoline hydrocarbon product. These process steps may either be integrated, producing an oxygenate intermediate, e.g. methanol or methanol dimethyl ether mixtures, which along with unconverted synthesis gas is passed to a subsequent step for conversion into gasoline or the process may be conducted in two separate steps with intermediate separation of oxygenates, e.g. methanol or raw methanol.

Useful oxygenates include methanol, dimethyl ether (DME) and higher alcohols and ethers thereof, but also oxygenates like ketones, aldehydes and other oxygenates may be applied.

Production of gasoline by the integrated process scheme is discussed in U.S. Pat. No. 4,481,305. Hydrocarbons and especially as gasoline are prepared by catalytic conversion in two subsequent reactors of a synthesis gas containing hydrogen and carbon oxides and having a mole ratio CO/H₂ below 1 and when the conversion commences a mole ratio CO/CO₂ of 5 to 20. Synthesis gas is converted with high efficiency in a first step into an oxygenate intermediate comprising predominantly dimethyl ether (DME) said mixture being converted in a second step into gasoline essentially according to the net reaction scheme

3H₂+3CO→CH₃OCH₃+CO₂+Heat (1)

CH₃OCH₃→2/n(CH₂)ₙ+H₂O+Heat (2)

(CH₂)ₙ represents the wide range of hydrocarbons produced in the gasoline synthesis step. After separation of the hydrocarbon product, unconverted synthesis gas comprising hydrogen and carbon oxides is recycled to the oxygenate synthesis step after CO₂ is at least partly removed, e.g. in a CO₂ wash.

U.S. Pat. No. 4,520,216A discloses a further process for synthetic hydrocarbons, especially high octane gasoline, from synthesis gas by catalytic conversion in two steps. In the first step the synthesis gas is converted to MeOH and/or dimethyl ether. In the second step the entire intermediate from the first step is converted to the synthetic hydrocarbons. The raw product stream from the second step is cooled and thereby separated into a condensed hydrocarbon product stream and a tail gas stream containing unconverted synthesis gas, the latter being recycled without further separation to the inlet of MeOH/DME synthesis step and here combined with fresh synthesis gas feed.

The tail gas stream separated from the raw product stream contains beside of the amount of carbon dioxide in the unreacted synthesis gas also the carbon dioxide being formed during the dimethyl ether synthesis by the above shown reaction (1).

In the known gasoline processes with a recycle of tail gas to the MeOH/DME synthesis, CO₂ builds up in the tail gas as it is an inert in the MeOH/DME synthesis and gasoline synthesis. High CO₂ concentrations even reduce the catalyst activity and inhibit the MeOH synthesis. The typical manner to remove CO₂ in a gas is by an acid gas removal process, in which acid gases such as CO₂ are removed from the gas streams. There are two types of acid gas removal processes: processes that use physical solvents (such as Rectisol using MeOH as solvent or Selexol using a mixture of glycols as solvent) and processes that use chemical solvents, such as amine-based solvents as in the MDEA process. The choice of CO₂ removal process depends on the gas composition, pressure and other parameters.

CO₂ removal processes are in general costly, both in capital and operating expenditure, and therefore avoiding a CO₂ removal altogether leads to cost savings.

The general objective of the invention is to provide an improved process scheme for the preparation of valuable hydrocarbons, boiling in the gasoline range, from carbon monoxide rich synthesis gas, by an intermediate oxygenate synthesis and a gasoline synthesis, whereby removal of carbon dioxide from a tail gas separated from the gasoline synthesis is not required. Instead of the costly CO₂ removal processes, a part of the tail gas from the gasoline synthesis is recycled to an autothermal reforming step in a synthesis gas preparation section in order to reduce the content of carbon dioxide from the recycled tail gas by reforming reactions.

This and other objectives of the invention are addressed by a process for the preparation of higher hydrocarbons boiling in the gasoline range from methane containing feed gas comprising the steps of

a) mixing the feed gas with a hydrogenated tail gas and autothermal reforming the mixed feed gas to a methanol synthesis gas containing hydrogen, carbon monoxide and carbon dioxide;
b) converting the methanol synthesis gas to a methanol and dimethyl ether containing effluent in presence of one or more catalysts active in the conversion of hydrogen and carbon oxides to methanol and dehydration of methanol to dimethyl ether;
c) converting the methanol and dimethyl ether containing effluent as prepared in step b) to a raw product containing hydrocarbons boiling in the gasoline range, water, unconverted methanol synthesis gas and carbon dioxide formed during the conversion of the methanol synthesis gas;
d) cooling and separating the raw product into a water fraction, a higher hydrocarbon fraction boiling in the gasoline range and into a tail gas with the unconverted methanol synthesis gas and the carbon dioxide;
e) hydrogenating a part of the tail gas as obtained in step d) to provide the hydrogenated tail gas; and
f) recycling the hydrogenated tail gas to step a).

The following embodiments can be combined with each other in any order:

Suitable feed gasses comprise natural gas, coke oven gas or blast furnace gas or combinations thereof.

In an embodiment of the invention the synthesis gas is produced from feed gas containing higher hydrocarbons, such as coke oven gas. The higher hydrocarbons contained in such gases must be converted to methane by means of a prereforming step prior to mixing the feed gas with the hydrogenated tail gas in step a).

In an embodiment of the invention the catalytic conversion of the methanol synthesis gas raw product in step (b) is carried out in the presence of a catalyst selected from the group consisting of oxides of Cu, Zn, Al and their mixtures, and combined with a solid acid.

In an embodiment of the invention the catalytic conversion of methanol and dimethyl ether containing effluent to the raw product in step (c) is carried out in the presence of a zeolite catalyst.

In an embodiment of the invention, the methanol synthesis gas has a molar ratio between hydrogen and carbon monoxide of less than 1.5 and a molar ratio between carbon monoxide and carbon dioxide of less than 10.

In a preferable embodiment of the invention, the synthesis gas has a molar ratio between hydrogen and carbon monoxide of approximately 1 and a molar ratio between carbon monoxide and carbon dioxide of approximately 1 to 4, thereby providing optimal conditions for gasoline synthesis.

Synthesis gas being useful for the invention is preferably adjusted to a H₂/CO ratio of about 1, and is reacted according to reactions (3), (4) and (5) in presence of an oxygenate catalyst including the known methanol catalysts e.g. catalysts with copper, zinc and/or aluminium oxide or their mixtures combined with a dehydration catalyst comprising a solid acid such as a zeolite, alumina or silicaalumina. The dehydration catalyst is useful for catalysing the dehydration of methanol to dimethyl ether (DME) according to reaction (5).

CO+2H₂ [symbol] CH₃OH (3)

CO+H₂O [symbol] CO₂+H₂ (4)

2CH₃OH [symbol] DME+H₂O (5)

The gasoline synthesis is performed at substantially the same pressure as employed in the oxygenate synthesis in the presence of a catalyst being active in the reaction of oxygenates to higher hydrocarbons, preferably C₅₊ hydrocarbons. A preferred catalyst for this reaction is the known zeolite H-ZSM-5.

It is a particular advantage of the process of the invention that it can accept a relatively high content of inert gases in the synthesis gas and even at moderate pressure provide a significant conversion of synthesis gas into gasoline via the oxygenate synthesis. The inerts comprising carbon dioxide and methane are carried through the entire gasoline synthesis steps and, eventually, end up in the tail gas stream from the gasoline synthesis step subsequent to the product separation.

The reaction of DME to higher hydrocarbons is known to be strongly exothermic and needs either indirect cooling (e.g. boiling water or fluidised bed reactor) or dilution of the reacting methanol synthesis gas.

In an embodiment of the invention a part of the tail gas is recycled to the conversion of dimethyl ether to gasoline in step c) in order to control the reaction temperature by dilution of the methanol and dimethyl ether containing effluent.

The oxygenate synthesis can be carried out at a temperature in the range of 200-300° C.

The MeOH/DME synthesis can be carried out at moderate pressures of approximately 4 MPa, but higher pressures of e.g. 8 to 12 MPa can be applied to increase the synthesis gas conversion and, in turn, the gasoline productivity.

Suitable operation pressures are in the range of 2-20 MPa, preferably 4-8 MPa. Preferably, a boiling water reactor or a gas cooled reactor can be used to provide cooling of the exothermic methanol/DME synthesis reaction.

The raw product from the gasoline reactor contains hydrocarbons in the range from C1 to C10, water and carbon dioxide and residual amounts of unconverted H₂, CO and inerts in the methanol synthesis gas.

By cooling and condensation a liquid phase of water, a liquid phase of mixed gasoline and light petroleum gas (LPG) is obtained, referred to as raw gasoline, is separated from a tail gas containing inerts, light hydrocarbons such as methane, ethane, etc. and carbon dioxide originating from the synthesis gas and additionally being formed in the upstream processes as described above. The raw gasoline may be further processed by conventional means to obtain a lower-boiling gasoline fraction and a fraction of LPG.

A part of the carbon dioxide containing tail gas can be recycled to the gasoline synthesis step for temperature control.

The process according to the invention does advantageously not require any separate upstream or intermediate carbon dioxide removal.

Still an advantage of the invention is that the amount of CO₂ being present in the synthesis gas feed stream and the amount of CO₂ being produced in the synthesis step may be recovered downstream the gasoline synthesis at essentially the synthesis pressure prevailing in the oxygenate synthesis step.

If a part of the tail gas is recycled to the gasoline reactor, the amount of recycled tail gas is adjusted to provide a MeOH/DME concentration inlet of the gasoline reactor between 2 and 10% by volume.

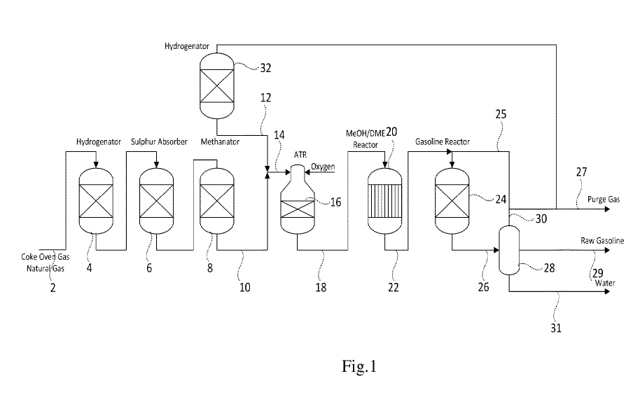

One embodiment according to the invention is illustrated in FIG. 1 showing a simplified flow sheet of a process for the preparation of gasoline from coke oven gas.

Synthesis gas is produced by feeding and passing a coke oven feed gas 2 containing beside of hydrogen and carbon oxides, methane and higher hydrocarbons through a hydrogenator 4 to hydrogenate sulphur compounds in the feed gas to hydrogen sulphide and a subsequent sulphur absorber 6 to reduce content of the hydrogen sulphide in the fed gas. The thus desulphurized feed gas is subjected to pre-reforming in methanator 8. In the methanator the higher hydrocarbons in the feed gas are cracked to methane. The thus treated feed gas 10 is mixed with a hydrogenated tail gas 12 recycled from a gasoline synthesis unit. The mixed gas stream 14 is converted to methanol synthesis gas 18 in an autothermal reformer 16 by a partial oxidation with oxygen and steam reforming reactions. The thus prepared methanol synthesis gas 18 is after cooling and removal of process condensate (not shown) introduced into a MeOH/DME reactor 20, preferably of the boiling-water type, charged with a catalyst system active in the conversion of synthesis gas into MeOH and DME according to the following reactions:

CO+2H₂ [symbol] CH₃OH (3)

CO+H₂O [symbol] CO₂+H₂ (4)

2CH₃OH [symbol] DME+H₂O (5)

to produce an effluent 22 comprising MeOH and DME. Effluent 22 from reactor 20 contains beside of MeOH and DME, unconverted synthesis gas and carbon dioxide contained in the synthesis gas and formed in the reaction of the gas to MeOH and DME. Effluent 22 is introduced into gasoline reactor 24. Prior to introduction into reactor 24, a part of a tail gas 30 from a downstream processing of the effluent from reactor 24 is admixed through line into effluent 25 in order to control temperature in gasoline reactor 24. In reactor 24 MeOH and DME are converted in presence of a catalyst as described above into predominantly C3-C10 hydrocarbons and water and withdrawn through line 26.

By cooling in a cooler (not shown) and condensation in condenser and separator 28, a raw gasoline fraction 29, a water fraction 31 and a tail gas 30 are obtained. Tail gas 30 contains CO₂, inerts and hydrogen together with carbon monoxide and additionally amounts of olefins.

A part of tail gas 30 is recycled to gasoline reactor 24 as discussed above. A further part of the gas is purged through line 27 to prevent build up of inerts in the synthesis loop. The remainder of tail gas 30 is recycled to the methanol synthesis gas preparation section and admixed into the methanated feed gas 10. Prior to admixing, the tail gas is hydrogenated in hydrogenator 32 in presence of a Cu/ZnO catalyst to reduce content of olefins in the tail gas.

### EXAMPLE

Raw gasoline is prepared by the above described process with reference to FIG. 1.

Process conditions and compositions of the various streams shown in the FIGURE are summarised in Table 1 below. The stream numbers in the Table refer to the stream numbers shown in the FIGURE.

TABLE 1

| | Temp. | Pressure | | |
|---|---|---|---|---|
| Stream | [° C.] | [bar g] | Flow | Composition [mole %] |
| 2 (coke | 40 | 29 | 30000 | CH4: 26.0, CO: 7.7, CO2: 2.1, Ethane: 1.6, |
| oven gas) | | | Nm3/h | H2: 58.4, Inerts: 3.3, O2: 0.3, Propane: 0.6 |
| 12 (recycle | 100- | 27 | 16700 | CH4: 1.6, CO: 11.2, CO2: 43.4, Ethane: 0.2, |
| to ATR) | 200 | | Nm3/h | H2: 32.5, Inerts: 7.9, Propane: 0.9, |
| | | | | Butane: 1.2, Water: 0.1, Aromatics: <0.1, |
| | | | | Paraffins: 0.2, Naphtenes: 0.1, Isoparaffins: 0.6 |
| 14 (inlet | 400- | 27 | 57700 | CH4: 18.9, CO: 4.1, CO2: 14.1, Ethane: 0.2, |
| to ATR) | 450 | | Nm3/h | H2: 30.4, Inerts: 4.0, Propane: 0.3, |
| | | | | Butane: 0.4, Water: 27.4, Aromatics: <0.1, |
| | | | | Paraffins: <0.1, Naphtenes: <0.1, Isoparaffins: 0.2 |
| 18 (outlet | | 23 | 86000 | CH4: <0.3, CO: 19, CO2: 8, |
| ATR) | | | Nm3/h | H2: 39, Inerts: 3, Water: 31 |
| 25 (recycle | 240 | 58 | 220000 | CH4: 1.6, CO: 11.2, CO2: 43.3, H2: 32.9, |
| to gasoline) | | | Nm3/h | Inerts: 7.8, Water: 0.1, Propylene: 0.2, |
| | | | | Aromatics: <0.1, Paraffins: 0.2, |
| | | | | Naphtenes: 0.1, Isoparaffins: 0.6, |
| | | | | Ethane: 0.1, Propane: 0.7, Butane: 1.2 |
| 27 (purge | 40 | 52 | 13700 | Same as 25 |
| gas) | | | Nm3/h | |
| 29 (raw | 40 | 52 | 8600 | Unit is wt %: CH4: 0.1, CO: 0.3, CO2: 19.1, H2: <0.1, |
| gasoline) | | | kg/h | Inerts: 0.3, Propylene: 0.3, Butane: 4.7, C5+: 75.2 |
| 31 (water) | 40 | 52 | 6700 | Unit is wt %: Water: 100 |
| | | | kg/h | |
| 32 (recycle | 40 | 52 | 16800 | Same as 25 |
| inlet hydro- | | | Nm3/h | |
| genator) | | | | |

The amount of purge gas in stream 27 without a recycle of tail gas to the ATR would be about twice the amount with the recycle.

1. Process for the preparation of higher hydrocarbons boiling in the gasoline range from methane containing feed gas comprising the steps of
a) mixing the feed gas with a hydrogenated tail gas and autothermal reforming the mixed feed gas to a methanol synthesis gas containing hydrogen, carbon monoxide and carbon dioxide;
b) converting the methanol synthesis gas to a methanol and dimethyl ether containing effluent in presence of one or more catalysts active in the conversion of hydrogen and carbon oxides to methanol and dehydration of methanol to dimethyl ether;
c) converting the methanol and dimethyl ether containing effluent as prepared in step (b) to a raw product containing hydrocarbons boiling in the gasoline range, water, unconverted methanol synthesis gas and carbon dioxide formed during the conversion of the methanol synthesis gas;
d) cooling and separating the raw product into a water fraction, a hydrocarbon fraction comprising higher hydrocarbons boiling in the gasoline range and into a tail gas with the unconverted methanol synthesis gas and the carbon dioxide;
e) hydrogenating a part of the tail gas as obtained in step d) to provide the hydrogenated tail gas; and
f) recycling the hydrogenated tail gas to step (a).

2. Process according to claim 1, wherein the feed gas contains higher hydrocarbons and wherein the feed gas is subjected to pre-forming prior to step (a).

3. Process according to claim 1, wherein the feed gas is composed of natural gas, coke oven gas and blast furnace gas or combinations thereof.

4. Process according to claim 1, wherein the catalytic conversion of the methanol synthesis gas in step (b) is carried out in the presence of a catalyst selected from the group consisting of oxides of Cu, Zn, Al and their mixtures, and combined with a solid acid.

5. Process according to claim 1, wherein the catalytic conversion of methanol and dimethyl ether containing effluent to the raw product in step (c) is carried out in the presence of a zeolite catalyst.

6. Process according to claim 1, wherein the methanol synthesis gas has a molar ratio between hydrogen and carbon monoxide of approximately 1 and a molar ratio between carbon monoxide and carbon dioxide of approximately 1 to 4.

7. Process according to claim 1, wherein a part of the tail gas is recycled to the conversion of dimethyl ether to gasoline in step (c).
